(19) United States
(12) Patent Application Publication
(10) Pub. No.: US 2006/0241640 A1
(43) Pub. Date: Oct. 26, 2006

(54) Device for dynamic tensioning of a natural or prosthetic knee joint

(76) Inventors: Louis Briard, Saint-Aignan (FR); Michael Breysse, Saint Pierre de Chandieu (FR)

Correspondence Address: YOUNG & THOMPSON, 745 SOUTH 23RD STREET, 2ND FLOOR, ARLINGTON, VA, 22202, US

(21) Appl. No.: 10/566,577

(22) PCT Filed: Jul. 29, 2004
(86) PCT No.: PCT/FR04/02046

§ 371 Date: May 31, 2006

(30) Foreign Application Priority Data
Aug. 5, 2003 (FR) 0309655

(51) Int. Cl. A61B 17/58

(52) U.S. Cl. 606/90

(57) ABSTRACT

The device includes at least one femoral insert (8A) with a condyle support surface (20A), for a bone or femoral implant, at least one tibial insert (10A), with a tibial plateau support surface (24A), for a bone or a tibial implant and force introduction element (4A, 30A), for application between the femoral insert and the tibial insert of a distraction force of a given intensity with the knee cap in place or not. The condyle support surface (20A) is dished in form and is provided with a sliding element (12A) for the bone or femoral implant when the knee joint is moved.

The present invention relates to a device for dynamic tensioning of a knee joint, also referred to as a device for distraction of the knee.

In the field of arthroplasty of the knee, the surgeon seeks to replace the damaged or deficient natural joint of the knee with a prosthetic joint which reproduces as faithfully as possible the kinematic properties of natural joints whilst forming a stable, durable and painless structure. The soft parts (capsule, ligaments and tendons) of the knee joint play a significant role in the mechanical strength of the joint when it is caused to move. However, these soft parts are specific to each patient and may be affected to a greater or lesser extent, for example, following illnesses. Furthermore, when prosthetic knee implants are positioned, the surgeon is often caused to excise specific ligaments, creating a new biomechanical environment.

It is therefore necessary, when a knee prosthesis is implanted, to evaluate the tension of these soft parts and, if necessary, to correct it in order to provide the best possible positioning for the prosthesis. More precisely, the desired object is to obtain tensions of the soft parts of the knee which are equal at 0 and 90° of flexion and which are maintained over the entire flexion arc of the prosthesis, satisfactory geometric alignment and extension without flexum in order to optimise the stresses in the upright position and provide the best possible fit with respect to the anatomy of the patient. A significant object is to obtain a good level of stability for the knee by means of an appropriate ligament balance.

To this end, a device for tensioning the soft parts is generally used, commonly referred to as “tensor” which generally comprises a femoral insert having two condyle support surfaces for the femur, a tibial insert having at least one support surface for the tibial plate, and means for applying, between the femoral and tibial inserts, a distraction force of a predetermined strength. Known means for measuring the relative positions of the femur and the tibia are associated with this tensor so that, by introducing the tensor in the space between the tibial end and the femoral articular end, it is possible to determine, under the selected tension value imposed by the tensor, the spacing between the tibia and the femur, as well as the angle HKA, that is to say, the angle taken internally between the femoral mechanical axis (defined by the centre of the hip and the centre of the knee) and the tibial mechanical axis (defined by the centre of the knee and the centre of the ankle), on the one hand, for extension and, on the other hand, in a state of flexion at 90°. Based on the measurements carried out in this manner, the surgeon selects the most appropriate constituent elements of the prosthesis, in particular from the set of elements which he has at his disposal.

However, it has been found that the use of tensors of this type does not always ensure optimum selection and/or positioning of the selected prosthetic elements, which does not allow optimum biomechanics to be achieved, in particular during retraction of the posterior soft parts of the knee in a state of flexum, and in the intermediate phases of flexion between 0 and 90°, and beyond 100°. The optimum biomechanics correspond to a “good tension” of the soft parts in all sectors of movement, that is to say, stability tension for the support zones and a micro level of frontal and rotational laxity clearance between 20 and 140°, allowing ease of movement without ever having hypertension or excessive or unequal laxity.

The object of the present invention is to provide a tensioning device which overcomes the above-mentioned disadvantages by allowing continuous control, that is to say, over substantially the entire flexion path of the knee joint, of the “good tension” of the soft parts.

To this end, the invention relates to a device for dynamic tensioning of a natural or prosthetic knee joint, of the type comprising a tensioning device for a natural or prosthetic knee joint, with or without a tibial cut being carried out, of the type comprising at least one femoral insert which has a condyle support surface for a femoral implant or bone; at least one tibial insert which has a support surface for a tibial plate for a tibial implant or bone; and means for applying, between the femoral and tibial inserts, a distraction force of a predetermined strength, with or without the kneecap being in position, characterised in that it is constructed so as to allow rotation of the joint and comprises means for maintaining the knee in a state of tension during rotation, and thus carrying out the measurements for various angles of rotation.

According to other features of this device, taken in isolation or according to all technically possible combinations:
the condyle support surface is in the form of a dish and is provided with sliding means for the femoral implant or bone when the knee joint is displaced;
the sliding means comprise juxtaposed rollers;
the sliding means comprise juxtaposed ball-bearings;
the condyle support surface is substantially cylindrical, having an axis which is substantially transverse relative to the direction-of distraction;
the maximum thickness of each femoral insert and tibial insert is less than or equal to 2.5 mm;
a femoral insert, and optionally a tibial insert, is/are provided for each inner and outer compartment of the knee joint;
it comprises means for measuring the spacing of the condyle support surfaces and tibial plate support surfaces, which means are capable of continuously measuring the spacing between the support surfaces when the knee joint is displaced;
it comprises means for measuring the distraction force between the femoral inserts and tibial inserts, which means are capable of continuously measuring the variation of the strength of the distraction force around the predetermined strength thereof when the knee joint is displaced;
the means for applying the distraction force comprise a force generation unit and a pair of branches which connect the generation unit to the femoral inserts and tibial inserts.

The invention will be better understood from a reading of the following description, given purely by way of example and with reference to the drawings, in which:

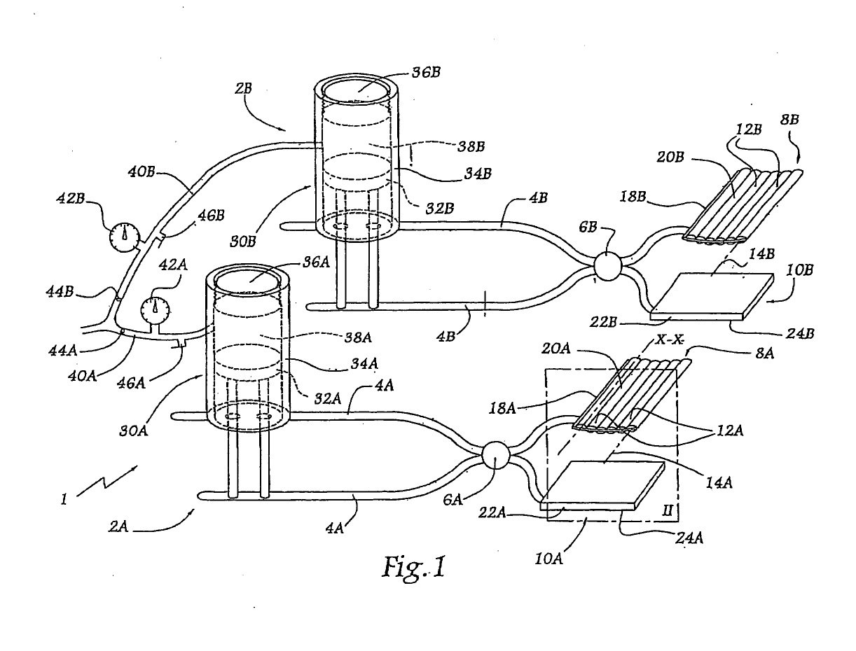

FIG. 1 is a perspective view of a tensioning device according to the invention,

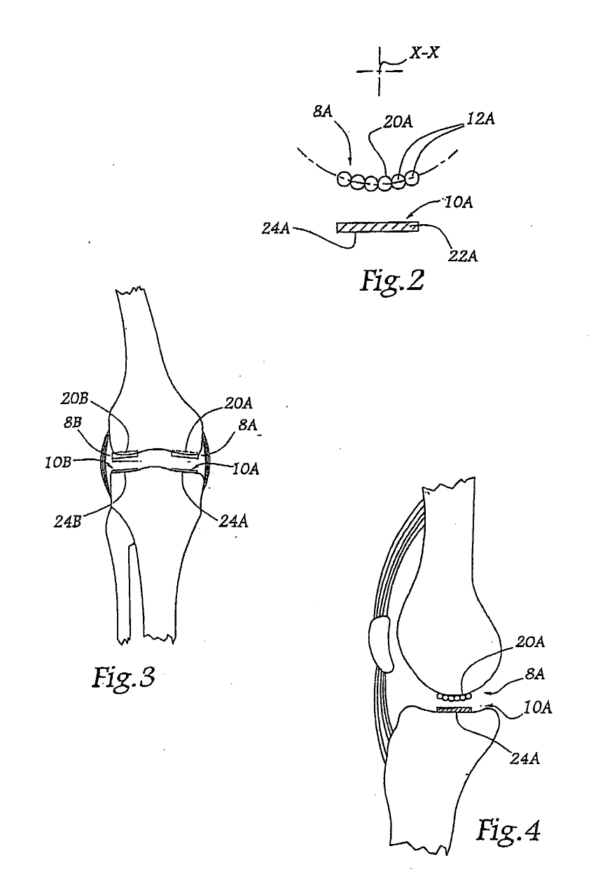

FIG. 2 is a section through plane II-II indicated in FIG. 1;

FIG. 3 is a schematic front view of a natural knee joint, at the inner side of which the femoral and tibial inserts of the device of FIG. 1, illustrated in section, are placed; and

FIG. 4 is a schematic lateral view corresponding to FIG. 3.

FIG. 1 illustrates a device 1 for tensioning a knee joint. This device 1 is substantially formed by two similar assemblies, that is to say, an inner assembly 2A for the inner compartment of the joint in the region of the inner femoral condyle, and an outer assembly 2B for the outer compartment (in the region of the outer femoral condyle). For convenience, in all of the following description, the device will be described and orientated with reference to a standard knee joint, the terms upper or top, lower or bottom, anterior or front, and posterior or rear, and the terms inner and outer corresponding to those used to commonly describe a joint of this type.

Furthermore, since the inner assembly 2A and outer assembly 2B comprise the same elements, only the elements of the inner assembly 2A will be described below, the corresponding elements of the outer assembly 2B being designated by the same number, followed by the letter B.

The inner assembly 2A is generally in the form of a pincer and comprises two branches 4A which are articulated relative to each other about a pivot axis 6A. The distal ends of the branches 4A are provided, respectively, with inserts (metal or non-metal), that is to say, a femoral insert 8A which is intended to be placed in contact with a lower end portion of the femur, in particular the condyle, and a tibial insert 10A which is intended to be placed in contact with an upper end portion of the tibia. The femoral and tibial inserts can be moved relative to each other and are particularly suitable for moving away from each other in accordance with a trajectory which is substantially in the form of an arc of a circle and which is centred on the articulation axis 6A when the proximal portions of the branches 4A are brought together.

More precisely, as illustrated in FIG. 2, the femoral insert 8A is generally in the form of a cylinder segment having an axis X-X which extends in a transverse direction. It comprises a series of juxtaposed rollers 12A which are mounted so as to rotate freely about axes 14A and which are, for example, fixedly joined to a common plate 18A which is rigidly connected to the corresponding branch 4A. The axes 14A extend substantially parallel with the axis X-X. The insert 8A thus provides a concave upper surface 20A in the form of a dish. This surface 20A is intended to form a support for the inner condyle of the femur, the radius of curvature of the dish 20A being selected so as to be close to the mean radius of curvature of this inner condyle in the sagittal plane. Of course, the rollers may be replaced with a fixed support surface.

The tibial insert 10A itself comprises a plate 22A having outer dimensions which are substantially similar to those of the femoral insert 8A. The plate 22A extends in a direction substantially parallel with the axis X-X of the femoral insert 8A. The tibial insert has, at the lower side, a surface 24A for supporting an inner tibial plate, that is to say, the natural inner upper surface of the upper end of the tibia relative to the inner condyle of the femur, or a substantially planar surface which is provided in this end of the tibia, for example, by means of a saw.

The femoral inserts 8A, 8B and tibial inserts 10A, 10B are thin, for example, in the order of 2.5 mm each, in order to be able to be slid between the femur and the tibia, with the knee cap advantageously not being in a dislocated state as illustrated in FIG. 3, in the region of each inner and outer compartment of the knee joint before the femur is cut. The inserts can be removable relative to the branches 4A and 4B and are fixed to these branches using rapid fixing means. The inserts are thus readily positioned in the condyle compartments whilst they are not yet connected to the branches of the device.

The proximal ends of the branches 4A of the inner assembly 2A are connected to each other by means of a unit 30A for generating a force which tends to bring together these ends. More precisely, the generation unit 30A comprises a piston 32A which is rigidly connected to one of the branches 4A, and a cylinder 34A which is rigidly connected to the other of the two branches 4A and at the inner side of which the piston 32A can move. The upper end of the cylinder 34A is provided with a screw 36A for sealed closure, delimiting, with the inner walls of the cylinder and the head of the piston 32A, a chamber 38A of variable volume. A pressurised fluid source, provided with control means which are not illustrated, is connected to this chamber 38A, via a connector 40A which is provided with a pressure gauge 42A, and control means 44A, 46A. This motor function carried out by this piston and this fluid may, in a variant, be carried out by an electric servomotor.

Advantageously, the device 1 comprises means which are not illustrated for measuring the spacing of the support surfaces 20A, 20B, 24A and 24B. These means, which are well known in the surgical field, comprise, for example, a high definition digital camera which is associated with an infrared emission source which covers the range in which a group of three markers are formed which passively reflect the infrared radiation. This group of three markers is positioned by the surgeon on one of the femoral or tibial portions of the joint, for example, on the lower portion of the femur, in order to form a three-dimensional marking system which allows the camera to determine in conventional manner the precise geometric location of one or more supplementary markers which are positioned on a bone portion which can be moved relative to the reference system of the first three markers, for example, positioned on the tibia. The surgeon is thus able, using appropriate calculation means, to determine, relative to the spatial reference system of the three markers which are positioned on the femur, the precise position of the tibia and, in particular, the angle of flexion between the two bones, the spacing between these bones, the lateral and anteroposterior displacements and the relative rotations.

The operation of the tensioning device is explained below, in the context of positioning a prosthetic knee joint.

The surgeon has a sterile set of implants of different sizes, each implant conventionally comprising: a tibial component formed by a base co-operating with a tibial rod in order to effectively seal the base on a cutting surface of the tibial plate, and having, for each type of base, a set of tibial plates, for example, of polyethylene, which can be attached to the base in order to provide a prosthetic tibial articular surface; a femoral component comprising a distal end which co-operates with a femoral rod which is intended for sealing in the femoral medullary canal, and having a prosthetic trochlea component which is intended to be articulated to the tibial plate, this trochlea component being either fixedly joined directly to the distal femoral end or, in other models, being able to be attached thereto, for example, with shims being interposed from a set of shims of variable thicknesses; the tibial component and the femoral component being connected or not in an articular manner by a pivoting means.

After positioning the various infrared markers, as explained above, then acquiring the anatomical forms of the relevant portions of the femur and the tibia and obtaining the precise anatomical model of these forms and dimensions using the calculation means mentioned above, the surgeon carries out, if necessary, a resection of the defective tibial plate and, using the movable marker, marks the position of this cutting plane.

Preferably, during the operation, the calculation means continuously provide the values of the current flexion and the angle HKA.

The surgeon then places the knee in a state of flexion, for example, at approximately 20°, and inserts the tensioning device 1 at the inner side of the joint. The support surface 20A is placed in contact with, or at least facing, the inner condyle of the femur, the support surface 20B is placed in contact with, or at least facing, the outer condyle of the femur and the support surfaces 24A and 24B are placed in contact with the natural tibial plate or the one which is obtained after resection. More precisely, by way of example, for each condyle compartment, a first insert is positioned, the second insert is then positioned after having dislocated the kneecap at the opposite side.

The force generation units 30A and 30B are activated so as to place the inner and outer compartments of the knee in a state of tension. For each compartment, a predetermined force is imposed, controlled by the pressure gauges 42A and 42B. The infrared markers allow the surgeon to verify that the spacing between the femoral and tibial portions of the knee joint is satisfactory. The simultaneous recording of the tensioning force and the spacing distance allows better calculation of the optimum tension of the soft parts. In the case of unsatisfactory femorotibial alignment, he relaxes or tightens the appropriate ligaments in order to move the femoral and tibial portions away from or towards each other.

Whilst holding the tensioning device 1 in position, the surgeon moves the knee into different positions of flexion and repeats these same measurements. Owing in particular to the concave form and the sliding properties of the condyle support surfaces 20A and 20B, the device 1 is stabilised relative to the knee during displacement. The surgeon brings the knee, for example, into the region of 0° of flexion, that is to say, into a state of extension. He continuously and intraoperatively verifies that, under the tension imposed by the support surfaces 20A, 20B, 24A and 24B, the relative spacing of the femoral and tibial portions is satisfactory.

In other words, the dynamic examination of the length, that is to say the tension, of the soft parts of the joint during the movements of flexion and extension allows the retraction diagnostics for these soft parts, in particular posterior parts, to be carried out and therefore allows the surgical actions for freeing or tightening these soft parts to be guided.

Advantageously, the integration of,the dynamic data on the one hand allows the anatomical centres of condyle rotation to be determined. Knowledge of these is necessary in order to decide the best positioning of the femoral implant. If the prosthetic centre of condyle rotation is offset or eccentric relative to the anatomical centre of rotation, stability, although correct at 0 and 90° of flexion, will be compromised for the intermediate angles, in particular at 45°, the soft parts being either too relaxed or too tight depending on whether the prosthetic centre is offset towards the front or the rear, or in the proximal or in the distal direction. On the other hand, the femorotibial alignment in the state of extension is measured correctly, the soft parts having correct tension. The surgeon, assisted by the above-mentioned calculation means, can measure this alignment in a dynamic manner between 0 and 120°, or even 150° of flexion of the knee using the device according to the invention. In the case of unsatisfactory alignment, surgical actions for freeing the soft parts are carried out and controlled intraoperatively using the device according to the invention.

Furthermore, once the femoral articular prosthesis of the knee is positioned, the device according to the invention allows the prosthetic femorotibial distances to be determined at a given pressure during the flexion/extension movements. The patient can thus be sure that the surgical procedure will be carried out correctly.

Other uses of the tensioning device 1 can also be envisaged:
once the device has been positioned on the natural or prosthetic joint, and is tensioned at a predetermined strength, the surgeon is able to examine the variations in pressure around the pressure value initially imposed, brought about by the movements of the femoral and tibial portions of the joint; these “response” variations are, for example, measured by the pressure gauges 42A and 42B or the sensors connected to the servomotor; the surgeon thus continuously verifies that the soft parts behave in the anatomically anticipated manner during flexion of the joint; advantageously, these measured values are transmitted to the, above-mentioned calculation means which determine the precise values of the tensions imposed by the soft parts; and
at the beginning, during or at the end of an operation, each assembly 2A and 2B can be used independently in order to determine the dynamic behaviour of each articular compartment; in this manner, if the differences in behaviour of the compartments deviate from the anticipated anatomical tolerances, the surgeon carries out adjustments for freeing or tightening the corresponding inner or outer ligament portions.

Furthermore, various arrangements and variants of the device described in detail above can be envisaged:
the sliding rollers 12A and 12B can be replaced with a series of juxtaposed ball bearings which are mounted so as to rotate freely or a smooth surface having a low friction coefficient, in order to allow the condyle surface to slide;
each force generation unit 30A and 30B can be replaced by a servomotor which-has a specific and adjustable force and which either allows a specific constant force to be generated, or, in the case of “response” measurement of the pressures applied by the femoral and tibial portions, allows the corresponding forces to be measured, or by a connector which is formed from shape memory metal;
the two tibial inserts 10A and 10B can be rigidly connected to each other so as to form a single tibial insert, the femoral inserts each being articulated relative to this single tibial insert, or optionally rigidly connected to each other; and/or
each pair of articulated branches 4A, 4B can be replaced with two arms which are substantially parallel with each other and which are provided, at the distal end thereof, with intra-articular inserts 8A and 10A, these two arms being able to be moved relative to each other in accordance with a translation movement which brings about the relative spacing of the intra-articular inserts, for example, by means of a motorised assembly having an endless screw, interposed between the two arms.

The device according to the invention can also be used as a tensor for the femoropatellar joint, the femoral insert being pressed against the trochlea surface and the tibial insert against the inner face of the kneecap. It is also possible to make provision for the dimensions and shapes of the inserts to be adapted for this purpose by conferring, in particular on the femoral insert, a convex form which complements the surface of the trochlea, or to have sets of inserts which can be assembled and disassembled on the arms of the tensor.

1. Device for dynamic tensioning of a natural or prosthetic knee joint, with or without a tibial cut being carried out, of the type comprising at least one femoral insert (8A) which has a condyle support surface (20A) for a femoral implant or bone; at least one tibial insert (10A) which has a support surface (24A) for a tibial plate for a tibial implant or bone; and means (4A, 30A) for applying, between the femoral and tibial inserts, a distraction force of a predetermined strength, with or without the kneecap being in position, characterised in that it is constructed so as to allow rotation of the joint and comprises means for maintaining the knee in a state of tension during rotation, and thus carrying out measurements for various angles of rotation.

2. Device according to claim 1, characterised in that the condyle support surface (20A) is in the form of a dish.

3. Device according to claim 1, characterised in that the condyle support surface (20A) is provided with sliding means (12A) for the femoral implant or bone when the knee joint is displaced.

4. Device according to claim 3, characterised in that the sliding means comprise juxtaposed rollers (12A).

5. Device according to claim 3, characterised in that the sliding means comprise juxtaposed ball-bearings.

6. Device according to claim 1, characterised in that the condyle support surface (20A) is substantially cylindrical, having an axis (X-X) which is substantially transverse relative to the direction of distraction.

7. Device according to claim 1, characterised in that the maximum thickness of each femoral insert (8A, 8B) and tibial insert (10A, 10B) is less than or equal to 2.5 mm.

8. Device according to claim 1, characterised in that a femoral insert (8A, 8B), and optionally a tibial insert (10A, 10B), is/are provided for each inner and outer compartment of the knee joint.

9. Device according to claim 1, characterised in that it comprises means for measuring the spacing of the condyle support surfaces (20A) and tibial plate support surfaces (24A), which means are capable of continuously measuring the spacing between the support surfaces when the knee joint is displaced.

10. Device according to claim 1, characterised in that it comprises means (42A) for measuring the distraction force between the femoral inserts (8A) and tibial inserts (10A), which means are capable of continuously measuring the variation of the strength of the distraction force around the predetermined strength thereof when the knee joint is displaced.

11. Device according to claim 1, characterised in that the means for applying the distraction force comprise a force generation unit (30A) and a pair of branches (4A) which connect the generation unit to the femoral inserts (8A) and tibial inserts (10A).

12. Device according to claim 2, characterised in that the condyle support surface (20A) is provided with sliding means (12A) for the femoral implant or bone when the knee joint is displaced.

13. Device according to claim 4, characterised in that the sliding means comprise juxtaposed ball-bearings.

14. Device according to claim 2, characterised in that the condyle support surface (20A) is substantially cylindrical, having an axis (X-X) which is substantially transverse relative to the direction of distraction.

15. Device according to claim 3, characterised in that the condyle support surface (20A) is substantially cylindrical, having an axis (X-X) which is substantially transverse relative to the direction of distraction.

16. Device according to claim 4, characterised in that the condyle support surface (20A) is substantially cylindrical, having an axis (X-X) which is substantially transverse relative to the direction of distraction.

17. Device according to claim 5, characterised in that the condyle support surface (20A) is substantially cylindrical, having an axis (X-X) which is substantially transverse relative to the direction of distraction.

18. Device according to claim 2, characterised in that the maximum thickness of each femoral insert (8A, 8B) and tibial insert (10A, 10B) is less than or equal to 2.5 mm.

19. Device according to claim 3, characterised in that the maximum thickness of each femoral insert (8A, 8B) and tibial insert (10A, 10B) is less than or equal to 2.5 mm.

20. Device according to claim 4, characterised in that the maximum thickness of each femoral insert (8A, 8B) and tibial insert (10A, 10B) is less than or equal to 2.5 mm.
